(19) United States
(12) Patent Application Publication
(10) Pub. No.: US 2019/0239752 A1
(43) Pub. Date: Aug. 8, 2019

(54) HYPERSPECTRAL IMAGING SYSTEM AND METHOD OF USING THE SAME

(71) Applicant: The Procter & Gamble Company, Cincinnati, OH (US)

(72) Inventors: Lucian DUMITRESCU, Deerfield Township, OH (US); Karen Kay KALLA, Cincinnati, OH (US); Dean Arthur ZIMMERMAN, Liberty Township, OH (US); Jaroslaw Pawel SACHA, West Chester, OH (US)

(21) Appl. No.: 16/264,732

(22) Filed: Feb. 1, 2019

Related U.S. Application Data
(60) Provisional application No. 62/625,788, filed on Feb. 2, 2018.

(51) Int. Cl. A61B 5/00; G01J 3/28; G01J 3/02; G06T 7/00; G06T 7/80; G16H 30/40

(52) U.S. Cl. CPC A61B 5/0075; G01J 3/2823; G01J 3/0229; A61B 5/441; A61B 5/7264; G06T 7/0012; G06T 2207/10152; G16H 30/40; G01J 2003/2826; A61B 2562/0233; A61B 2576/02; G06T 2207/30088; G06T 2207/10048; G06T 7/80

(57) ABSTRACT

A hyperspectral imaging system that includes an image capture device, an illumination component, a tunable filter, and an infrared cut-off filter. The hyperspectral system can capture a spectral image of a target object such as a clinical test subject across a spectral range of at least 450 nm to 700 nm at a spectral resolution of at least 50 nm. The infrared cut-off filter is positioned between the target object and the tunable filter to reduce leak-through and improve the performance of the hyperspectral imaging system.

### FIELD

The present disclosure relates generally to a hyperspectral imaging system. More specifically, the present disclosure relates to a hyperspectral imaging system with suitable operating speed and imaging size for use in diagnosing and/or evaluating skin conditions on the face of person in a clinical test setting.

### BACKGROUND

There are numerous cosmetic skin care compositions available for treating a wide variety of skin conditions (e.g., hyperpigmentation, fine line and wrinkles, oiliness, and dryness). When evaluating the effectiveness of a skin care composition, it is not uncommon for a manufacturer to test a composition in a clinical setting. When testing a skin care composition in a clinical setting, test subjects and test administrators generally prefer non-invasive test methods such as imaging techniques or visual evaluations over invasive methods such as a biopsy. Image analysis techniques typically involve capturing an image of a portion of skin (e.g., in a photograph) and then analyzing the captured image, for example, to evaluate the presence or severity of a skin condition of interest or evaluate the presence or efficacy of a skin care composition or skin care agent. Common image analysis techniques include evaluation by an expert skin grader and evaluation by a computer using computer vision and/or computer learning techniques.

When capturing an image of a human subject in a clinical setting, it can be important for the subject to remain still, especially when a longer exposure time (i.e. slower camera shutter speed) is used. Movement by the subject may reduce the quality of the captured image (e.g., cause blurriness), and in situations where longer exposure times are used, the impact of movement on image quality can be exacerbated. Thus, it would be desirable to have a system capable of capturing a relatively large image area (e.g., an entire face) in about five seconds or less, to minimize the time a test subject must remain still.

There are a variety of imaging techniques known for use in evaluating skin in a clinical setting. For example, U.S. Pat. No. 6,571,003 describes a system that utilizes a digital camera to capture an image of a subject's face. The captured image is subsequently analyzed by a computer to identify cosmetic skin defects such as red spots. The system can then visually display the identified defects on a display device. While such a system may be useful for identifying visible defects in skin, the spectral bands in which the system operates may be limited to just a few bands in the visible spectrum (e.g., red, green, and blue), which in turn may limit the types and/or severity of defects that can be analyzed.

In some instances, multispectral imaging may be used to provide additional spectral bands for non-invasively analyzing skin. For example, the multispectral imaging system described in U.S. Pat. No. 7,603,031 may provide additional spectral modalities by employing various combinations of lighting characteristics and filter combinations. However, multispectral imaging techniques still may not provide a desired number of contiguous spectral bands (e.g., 10 or more), and manipulating the number of filters and/or lights to provide the desired number of spectral bands may result can be cumbersome when capturing images of a test subject in a clinical setting.

Conventional hyperspectral imaging devices such as commercially available spectrophotometers are commonly used to capture and record images of a target surface in contiguous spectral bands across a predetermined range. The recorded spectral images typically have a relatively fine spectral resolution across a relatively wide spectral range. For example, a conventional hyperspectral imaging system may analyze a spectral range of 400 nm to 800 nm in 10 nm bands, thereby providing 40 contiguous spectral bands. However, spectrophotometer-based systems such as the one disclosed in U.S. Pat. No. 8,761,476 are not be suitable for analyzing large sample areas (e.g., areas of greater than 50 cm² or greater than 100 cm²) or providing suitable spatial resolution because the output may include “spectral averaging.”

Other hyperspectral systems such as the one described in U.S. 2007/0237374, focus on specific skin defects that can be detected using a narrow range of spectral bands, but these systems do not address the challenges associated with analyzing skin defects over an entire range of spectral bands.

Accordingly, there remains a need for a hyperspectral imaging system suitable for analyzing skin in a clinical setting, which provides high speed image capture capability, suitable spectral resolution, suitable spatial resolution, large image acquisition area, and high throughput image processing capability.

### SUMMARY

Disclosed herein is a hyperspectral imaging system comprising an image capture device, an illumination component, a tunable filter, and an infrared cut-off filter positioned between a target object and the tunable filter. The image capture device is positioned to capture an image of a target object. The illumination component is configured to illuminate the target object with a sufficient amount of light for the hyperspectral imaging system to capture suitable spectral images of the target object and generate a hyperspectral image. Also disclosed herein are method of using the hyperspectral system, including for determining a characteristic of a skin condition.

### BRIEF DESCRIPTION OF THE DRAWINGS

FIG. 1 illustrates the IR cutoff response of an exemplary IR cutoff filter.

FIGS. 2A, 3A, 4A, and 5A illustrate the amount of light transmitted through a tunable filter at 400 nm, 410 nm, 420 nm, and 430 nm, respectively.

FIGS. 2B, 3B, 4B, and 5B illustrate the amount of light transmitted through a tunable filter in combination with an IR cutoff filter at 400 nm, 410 nm, 420 nm, and 430 nm, respectively.

FIG. 6 illustrates individual spectral images in 10 nm spectral bands across a range of 410 nm to 720 nm.

FIG. 7 illustrates a hyperspectral cube formed from individual spectral images.

FIG. 8 depicts an example of a hyperspectral imaging system.

FIGS. 9A and 9B depict an example of a hyperspectral imaging system.

### DETAILED DESCRIPTION

The hyperspectral imaging system herein enables a user to more quickly and conveniently capture, store, and/or analyze a hyperspectral image of a test subject in a clinical setting, as compared to conventional hyperspectral imaging capture systems. The present hyperspectral imaging system provides a relatively large image acquisition area, compared to conventional hyperspectral imaging systems, which enables a user to capture a spectral image of the entire face a test subject (or other body portion such as an arm, leg, back, chest, buttock, or armpit) in a single image. The present system also provides excellent spectral and spatial resolution, for example, by providing at least 10 spectral bands and reducing or eliminating color averaging. The present system includes an infrared (“IR”) cut-off filter to improve signal quality, and thus image quality, at the blue end of the electromagnetic spectrum (e.g., 400 nm-450 nm). The present system provides a fast image acquisition time and fast total acquisition time to provide high test subject throughput, which is highly desired in a clinical setting.

As used in the description and the appended claims, the singular forms “a,” “an,” and “the” are intended to include the plural forms as well, unless the context clearly indicates otherwise. The number of significant digits conveys neither a limitation on the indicated amounts nor on the accuracy of the measurements. All numerical amounts are understood to be modified by the word “about” unless otherwise specifically indicated. All measurements are understood to be made at 25° C. and at ambient conditions, where “ambient conditions” means conditions under about one atmosphere of pressure and at about 50% relative humidity. All numeric ranges are inclusive of narrower ranges and combinable; delineated upper and lower range limits are interchangeable to create further ranges not explicitly delineated.

The hyperspectral system herein can comprise, consist essentially of, or consist of, the essential components as well as optional components described herein. As used herein, “consisting essentially of” means that the system or component may include additional components, but only if the additional components do not materially alter the basic and novel characteristics of the claimed system or method.

“About,” as used herein, modifies a particular value by referring to a range equal to the particular value plus or minus twenty percent (+/−20%) or less (e.g., less than +/−15%, +/−10%, or even less than +/−5%).

“Cosmetic composition” means a composition that is intended to provide a desired visual effect on an area of the human body. The visual cosmetic effect may be temporary, semi-permanent, or permanent. Some non-limiting examples of cosmetic compositions include products that leave color on the face, such as foundations, concealers, and the like, and compositions that regulate and/or improve a skin condition (“skin care compositions”), such as skin moisturizers, fines line and wrinkle treatments, hyperpigmentation treatments, and skin barrier function treatments. Some non-limiting examples of skin care compositions are described in U.S. Publication Nos. 2008/0206373 and 2010/0189669.

“Coupled” means two components are joined to one another, either directly or indirectly, e.g., via a third component, such that the coupled components are in mechanical, electrical, and/or electronic communication with each other.

“Exposure time” refers to the amount of time that the imaging sensor of an image capture device is exposed to light when capturing an image. For example, the exposure time of a digital camera is typically determined by the shutter speed of the camera.

“Hyperspectral image” refers to a set of 10 or more images of the same target object captured at different wavelengths in a single image capture session.

“Hyperspectral image stack” refers to a hyperspectral image arranged in a cube or cube-like structure having two spatial dimensions (x,y) and one spectral dimension (λ).

“Image acquisition time” means the time it takes to capture and store an image at a particular wavelength or spectral band.

“Light” herein refers to electromagnetic radiation having a wavelength of between 380 nm and 750 nm.

“Skin Condition” means a condition that undesirably affects the health, appearance, and/or feel of one or more layers of skin. Some non-limiting examples of skin conditions include conditions that reduce the thickness of one or more layers of skin; reduce the elasticity or resiliency of skin; reduce the firmness of skin; increase the oily, shiny, and/or dull appearance of skin; reduce the hydration status or moisturization of skin; increase the appearance of fine lines and/or wrinkles; reduces skin exfoliation or desquamation, decreases skin barrier properties, worsens skin tone, increases the appearance of redness or skin blotches; and/or reduces the brightness, radiancy, or translucency of skin. Other non-limiting examples include skin conditions associated with or caused by inflammation (e.g., red spots associated with acne), irritation, enlarged pores, clogged pores, sun damage, and/or ageing (intrinsic or extrinsic), such as hyperpigmentation (e.g., age spots), seborrheic keratosis, actinic keratosis, UV exposure, skin sallowness or yellowness, sebum secretion, rough texture, wrinkles, compromised skin barrier (e.g., dry skin), contact dermatitis, atopic dermatitis, eczema, keratinization disorders, psoriasis, wound healing, combinations of these and the like.

“Spectral band” refers to a range of wavelengths having a defined upper and lower limit. For example, a spectral band with a bandwidth of 10 nm may include any wavelength between 401 nm and 410 nm.

“Spectral image” refers to an image generated by an image capture device using light passed thru a filter that is tuned to a specific wavelength or spectral band.

“Spectral resolution” refers to the finite, distinct wavelength interval that the system can separate light into and still distinguish the wavelengths from each other.

“Total acquisition time” refers to the amount of time it takes the system to capture and store all the images across the selected wavelength range. For example, if images are captured at intervals of 10 nanometers (“nm”) across a range of 420 nm to 730 nm, then the total acquisition time would be the time it takes to capture and store all 32 images.

### Hyperspectral Imaging System

The hyperspectral imaging system described herein includes an image capture device, an illumination source, a tunable filter, and an IR cutoff filter. The present system may, optionally, include a control component and a processing component. The control component may be used to control some or all of the system hardware, and the processing component may be used to calibrate the system and/or analyze captured images. Some or all of the various components of the present system, which are described in more detail below, may be in electronic communication with one another, for example, via a wired or wireless network.

The present system may have a total acquisition time of 5 seconds or less (e.g., less than 4, 3, 2, or even less than 1 second), but typically greater than 50 ms. Image acquisition time at a particular wavelength may vary, based on how much light the filter(s) in the system is able to pass. For example, it is not uncommon for a LCTF to pass less light at the blue end of the spectrum, which translates to a longer acquisition time to collect a suitable amount of light for an image.

The present system may have a spectral resolution of 50 nm or less (e.g., less than 40, 30, or 20 nm, or even less than 10 nm, but typically 1 nm or more) across a spectral range of at least 450 nm to 700 nm (e.g., 420 nm to 710 nm, 410 nm to 730 nm, or even 380 nm to 750 nm). The present system should also have a resolution of at least 640×480 pixels, a flexible acquisition area, and a high throughput capability for image acquisition and, optionally, processing. In some instances, the hyperspectral system may include a stable mounting platform to facilitate the image capture process in a clinical environment.

The hyperspectral imaging system herein includes an image capture device such as, for example, a digital camera or the like, which includes an imaging sensor for receiving light and transforming the received light into a digital image. In some instances, the image capture device can capture and store digital images at a resolution of at least 640×480 pixels (e.g., at least 1000×800 pixels, 1200×1000 pixels, 1500×1300 pixels, or even at least 2000×2000 pixels). In some instances, the image capture device generates digital images that have a pixel size of 100 μm or less (e.g., 50 μm, 40 μm, 30 μm, or even 20 μm or less), but typically greater than 1 μm, 5 μm, 10 μm, or 15 μm. In some instances, the image capture device can capture at least 10 images per second (e.g., 15, 20, 25, or more images per second) and has a variable exposure range, for example, between 0.04 ms and 2 seconds. The image capture device may be configured to transfer captured images to another device or component of the system (e.g., a local or remote computer or memory storage device that is in electronic communication with the image capture device) for storage and/or processing. Nevertheless, it may be desirable to provide the image capture device with sufficient storage capacity (e.g., camera image buffer or secure digital (“SD”) memory card) to store at least 10 images (e.g., 30, 40, 50, or 100 or more images). A non-limiting example of a suitable image capture device for use herein is a Grasshopper3™ U3, available from Point Grey Research, Inc., Canada, or an equivalent thereto.

The image capture device may include one or more lenses removably or permanently joined to the image capture device. The lens may be a high-resolution, high-speed lens configured to focus light on an imaging sensor (e.g., a CCD or CMOS type imaging sensor). In some instances, the lens may include a filter or coating to selectively reduce the intensity of certain wavelengths of light detectable by the sensor. In some instances, it may be desirable to use an achromatic lens that has a field of view sized to minimize or eliminate optical vignetting, which can reduce the quality of subsequent image processing and analysis techniques performed by the system. An achromatic lens will typically limit the effects of chromatic and spherical aberration by bringing two or more wavelengths of light (e.g., red and blue) into focus on the same plane. But if the field of view is too large, then undesirable vignetting may result. The distance and/or angle between the lens and the target object (e.g., test subject's face) may be adjusted as desired, as long as a sufficient amount of light reaches the imaging sensor to provide an image of suitable quality at each wavelength selected. A non-limiting example of a suitable lens for use in the present system is an Apo-Xenoplan 2.8/50, available from Schneider Optics, Inc. Hauppauge, N.Y., or an equivalent thereto.

The hyperspectral imaging system herein includes a tunable filter coupled to the image capture device. Tunable filters are filters that can be manually and/or automatically adjusted to pass light of a certain wavelength or spectral band while inhibiting the passage of light at other wavelengths. The tunable filter may be mechanically joined to the lens of the image capture device (e.g., via mated threads, snaps, clamps, screws, and the like) such that light passes through the filter prior to passing through the lens to the imaging sensor of the image capture device. The tunable filter may have a spectral resolution of at least 50 nm across a spectral range of 380 nm to 750 nm. For example, the tunable filter may have a spectral resolution of 1, 2, 3, 4, 5, 10, 15, 20, 25, 30, 35, 40, 45, or even 50 nm across a spectral range of 400 nm to 740 nm, 410 nm to 730 nm, 420 nm to 720 nm, 430-710, or even 450 nm-700 nm. The tunable filter should be tunable to at least 10 different wavelengths or spectral bands within the spectral range of the tunable filter.

In some instances, the tunable filter is a liquid-crystal tunable filter (“LCTF”). An LCTA may be particularly suitable for use in the present system because they can be electronically controllable, typically contain no moving parts, and can provide rapid, vibrationless wavelength selection in the filter's spectral range. The LCTF may also include one or more polarizing filters to polarize (e.g., cross-polarize) light entering the tunable filter, for example, to reduce the shine effect that sometimes occurs when capturing images of human skin. A non-limiting example of a suitable LCTF is a VariSpec™ brand LCTF, available from Cambridge Research & Instrumentation, Inc., Boston, Mass.

In some LCTFs, light at unselected wavelengths may be undesirably transmitted through the filter. This undesired light is sometimes referred to as “leak-through”. It is believed, without being limited by theory, that leak-through is a result of natural limitations of the liquid crystal elements in the tunable filter at certain wavelengths. For example, it has been found that when an LCTF is tuned to transmit light at wavelengths of between 400 and 430 nm, it may also allow some light in the near-IR spectra (i.e., 700 to 730 nm) to pass. When light of an undesired wavelength passes through the tunable filter along with light of the desired frequency, it increases the total amount of light (i.e., photons) detected by the imaging sensor. As a result, the sensor may not be able to distinguish light at the desired wavelength from light at the undesired wavelength. Since light at the undesired wavelength is essentially “noise” to the system, the signal-to-noise ratio (“S/N”) is lower at the desired wavelength, which reduces the accuracy of the system to measure the amount of light reflected off the target object at the desired wavelength.

To reduce the amount of leak-through light, it can be important to include an IR cut-off filter in the present system. It may be particularly desirable to include an IR cutoff filter that reduces the intensity of (or eliminates entirely) light at wavelengths of between 700 and 750 nm (“near-IR”). In some instances, the IR cutoff filter is configured to reduce the intensity of near-IR by 50% or more (e.g., 60%, 70%. 80%, or even 90%). The IR cutoff filter may be placed before or after the tunable filter, as long as it reduces the intensity of near-IR light reaching the imaging sensor. In some instances, the tunable filter may also include a separate IR filter that acts to protect the tunable filter from heat damage associated with excessive IR light passing thru the filter. However, these thermal protectant IR filters generally do not provide the desired level of IR attenuation needed to improve system accuracy.

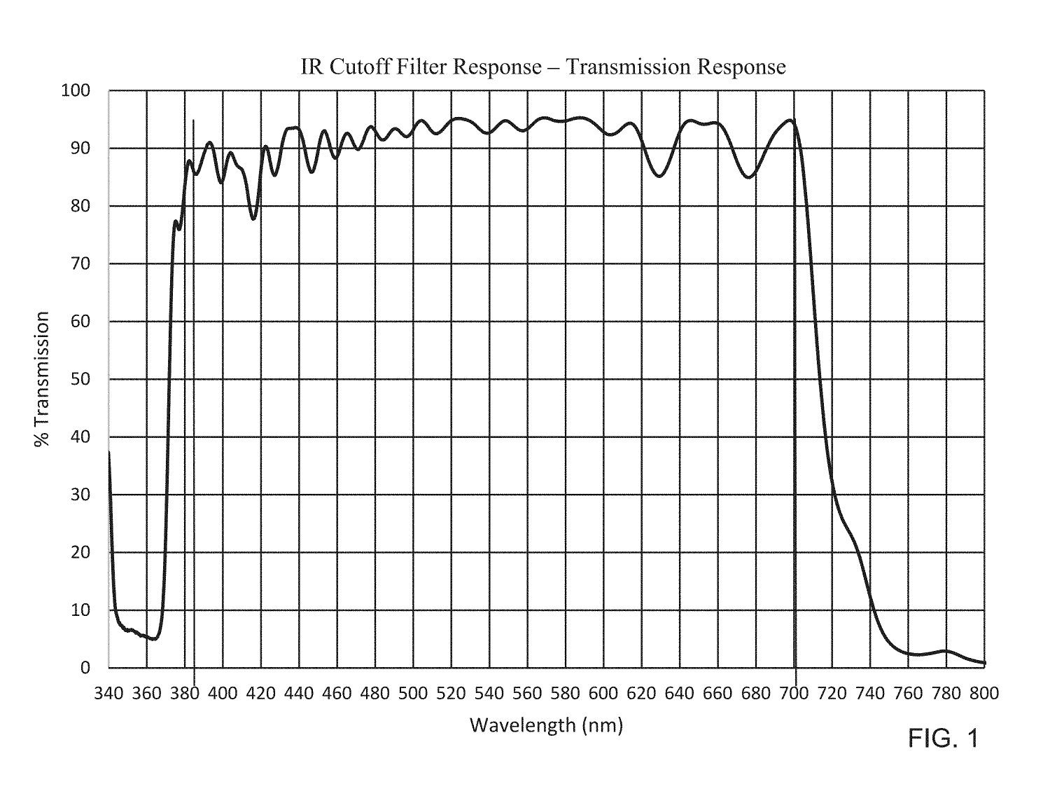

FIG. 1 illustrates a suitable example of an IR cut-off filter response. As illustrated in FIG. 1, the filter substantially reduces the passage of light at wavelengths greater than 700 nm. The filter in this example is a hot mirror filter available from the Tiffen Company, Hauppauge, N.Y.

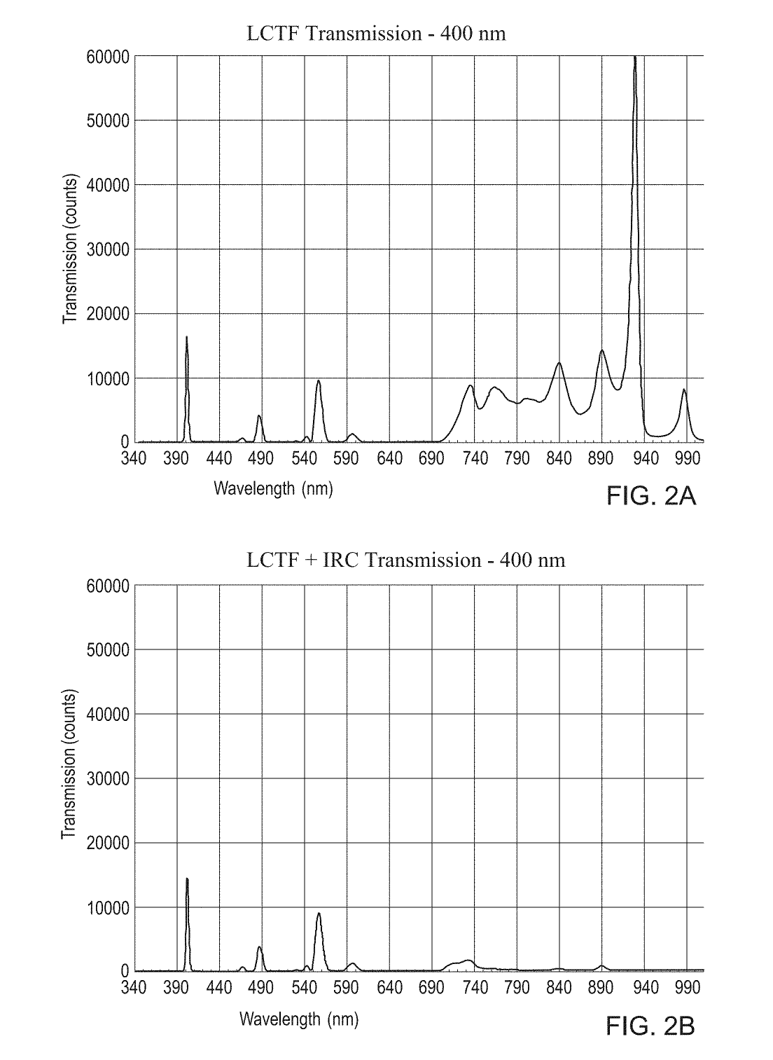

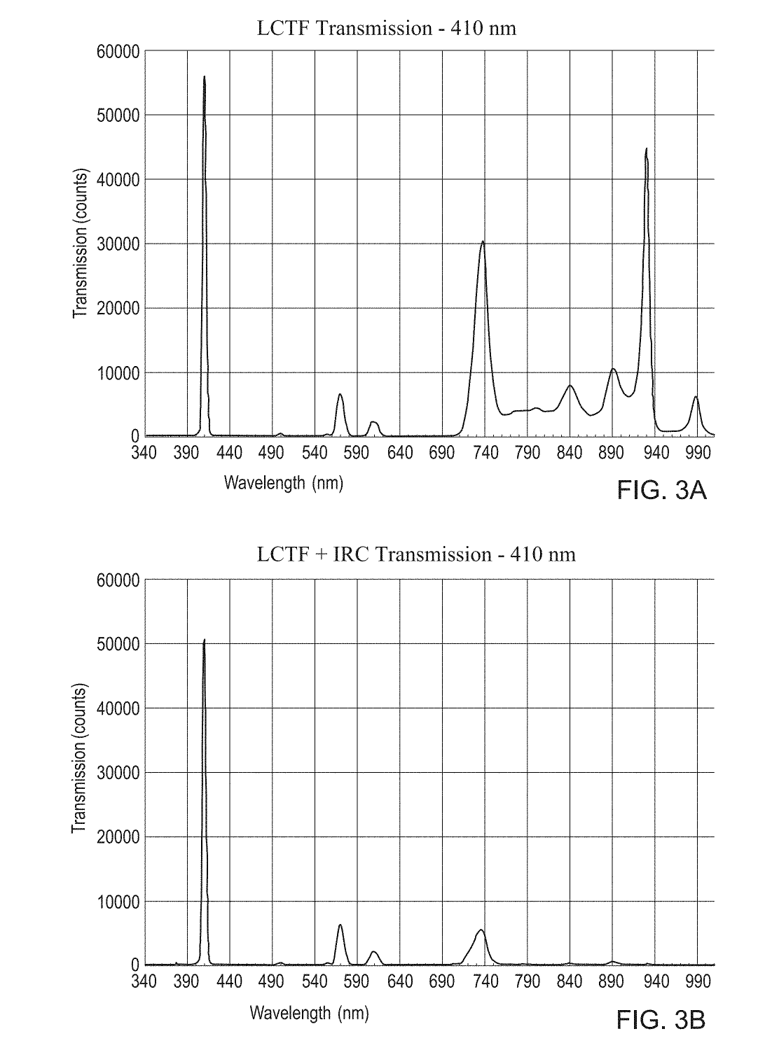

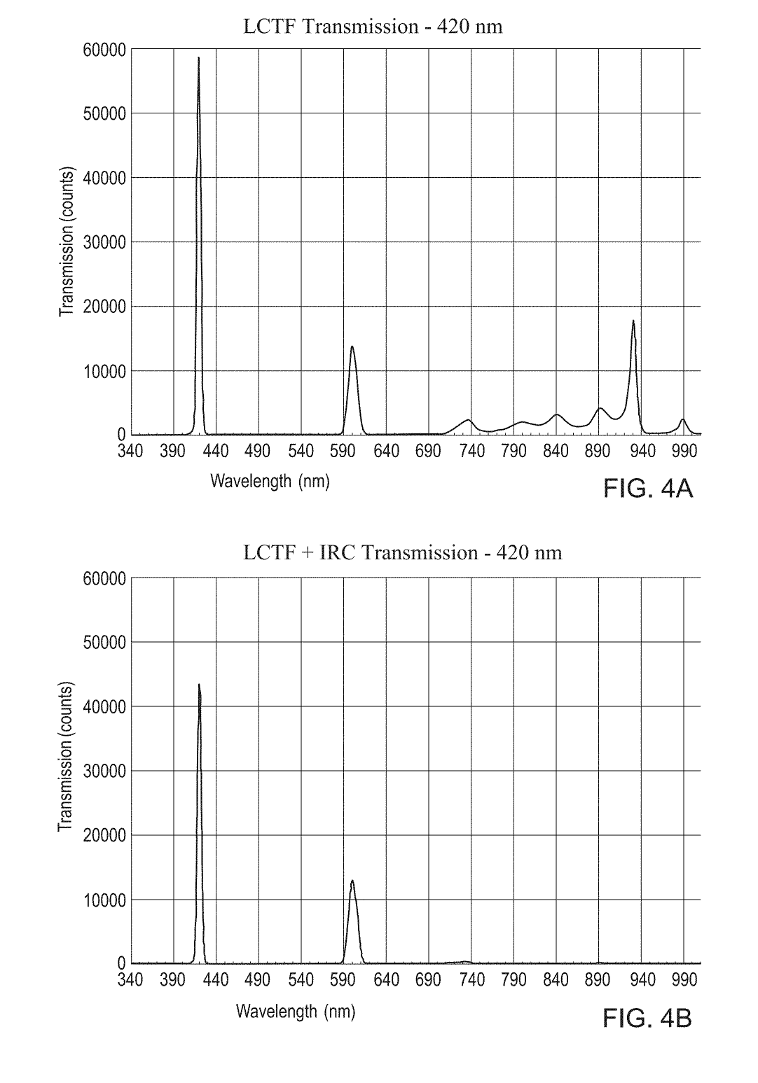

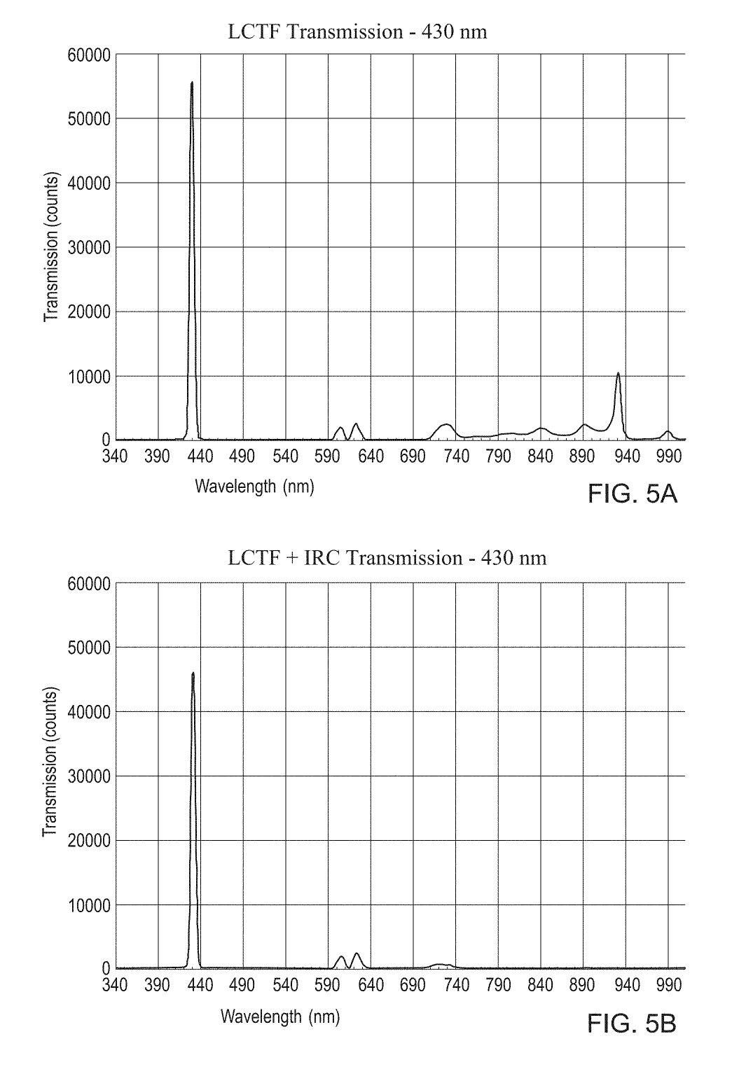

FIGS. 2-5 illustrate a comparison of light transmission through an LCTF with and without an IR cutoff filter. FIGS. 2A, 3A, 4A, and 5A illustrate the amount of light passing through the LCTF without an IR cutoff filter at 400 nm, 410 nm, 420 nm, and 430 nm, respectively. FIGS. 2B, 3B, 4B, and 5B are counterparts to FIGS. 2A, 3A, 4A, and 5A, respectively, and illustrate the amount of light passing through an LCTF when an IR cutoff filter is placed in front of the LCTF (i.e., the light passes through the IR cutoff filter before it passes through the LCTF). The IR cutoff filter illustrated in FIGS. 2B, 3B, 4B, and 5B has the same IR transmission response as shown in FIG. 1, and the LCTF includes a built-in IR cut-off filter.

By comparing the transmission profiles in each of FIGS. 2A-5A to its counterpart in FIGS. 2B-5B, it can be seen that a significant amount of light from unselected wavelengths (i.e., >700 nm) passes through the filter as noise. Since the present system may not be able to distinguish transmitted light at the desired wavelength from transmitted light at the undesired wavelengths, the S/N ratio may be too low to provide a useful spectral image. However, when a suitable IR cutoff filter is used in combination with the LCTF, the amount of noise is significantly reduced, and the system can provide a more suitable spectral image.

The illumination component of the present system includes one or more light sources capable of providing a suitable amount of light at the desired spectra (e.g., 380 nm-750 nm) on a target object (e.g., the face or other portion of the body of a test subject). It is to be appreciated that the farther away the target object is from the camera, the more intense the light source may need to be to provide sufficient light intensity at each wavelength; this may be especially noticeable at shorter wavelengths. A continuous light source that provides a relatively uniform spectral distribution is preferred. In some instances, a power source that minimizes the amount of flicker caused by standard alternating current power sources may be used (e.g., a medical grade power sources).

The source(s) of light may be positioned behind the tunable filter to reduce or eliminate stray light noise from entering the filter directly from the light source(s). It may also be desirable to position the light source(s) such that the light is evenly distributed on the target object, and thus is more likely to reflect evenly toward the image capture device. For example, the illumination component may include 2 or more light sources (e.g., 3, 4, 5, 6, 7, 8, or more) arranged equidistantly from one another around the image capture device. The light source(s) may include any suitable type of light source known in the art (e.g., light emitting diodes (LED), fluorescent, sodium, metal halide, halogen, neon, incandescent, high intensity discharge, and combinations of these). A non-limiting example of a suitable light source is an AR111 (18.5 W) halogen light, available from Soraa Inc., Fremont, Calif. In some instances, the illumination component may include two or more different types of light sources and/or different intensities of light to provide the desired intensity across a selected spectrum. For example, the illumination component may include a first light source that provides a suitable light intensity at 430 to 720 nm and a second light source that provides a suitable light intensity at 400 to 430 nm.

In some instances, the illumination component may include one or more polarization filters to cross polarize the light emitted from the light source. Polarizing the light may help reduce the shine effect sometimes observed when photographing human skin. The polarizers may be positioned to cover all or a portion of one or more of the light source(s) that make up the illumination component. It is to be appreciated that polarizers may be positioned at any position between the light source(s) and the tunable filter, as desired. In some instances, it may be desirable to configure the polarizers of the illumination component to function cooperatively with the optional polarizers of the tunable filter, when included, to yield the desired degree of light polarization.

The present system may include a positioning system that enables a user to adjust the position of the image capture device in at least one plane relative to the position of the target object or test subject. It may be desirable for the positions system to minimize or eliminate movement and/or vibration from the test subject and/or system components during the intended use of the system. In some instances, the positioning system may provide a level, stable platform that supports the image capture device, other system components, and/or a portion of a test subject's body. For example, the positioning system may include vertical and/or horizontal mounting elements that join the image capture device to a stable surface such as a table top. The vertical and horizontal mounting elements may enable a user to reposition the image capture device (e.g., manually or automatically) while ensuring that the image capture device remains secured to the mounting element(s). Suitable methods and devices for automatically or manually repositioning a camera secured to a vertical or horizontal mounting element are known in the art.

In some instances, the hyperspectral imaging system herein includes an image acquisition control component for controlling one or more aspects of image acquisition. For example, the image acquisition control component may include hardware, firmware, and/or software for operating the camera and/or tunable filter, causing images to be displayed on a display device (e.g., real time images and/or raw or processed images), adjusting the position of the positioning system, generating hyperspectral image stacks, and/or saving images and data to a non-transitory computer readable medium. The image acquisition component software may include control logic stored on a non-transitory, computer-readable storage medium such as, for example, random access memory (SRAM, DRAM, etc.), read only memory (ROM), registers, and/or other forms of computing storage hardware, which may be part of the image capture device, a remote computing device, and/or another component of the hyperspectral imaging system. The image acquisition control component hardware may include circuitry, and/or other computing infrastructure, which enables the image acquisition control component and/or control logic to communicate electronically with other components of the hyperspectral imaging system via a wired or wireless connection.

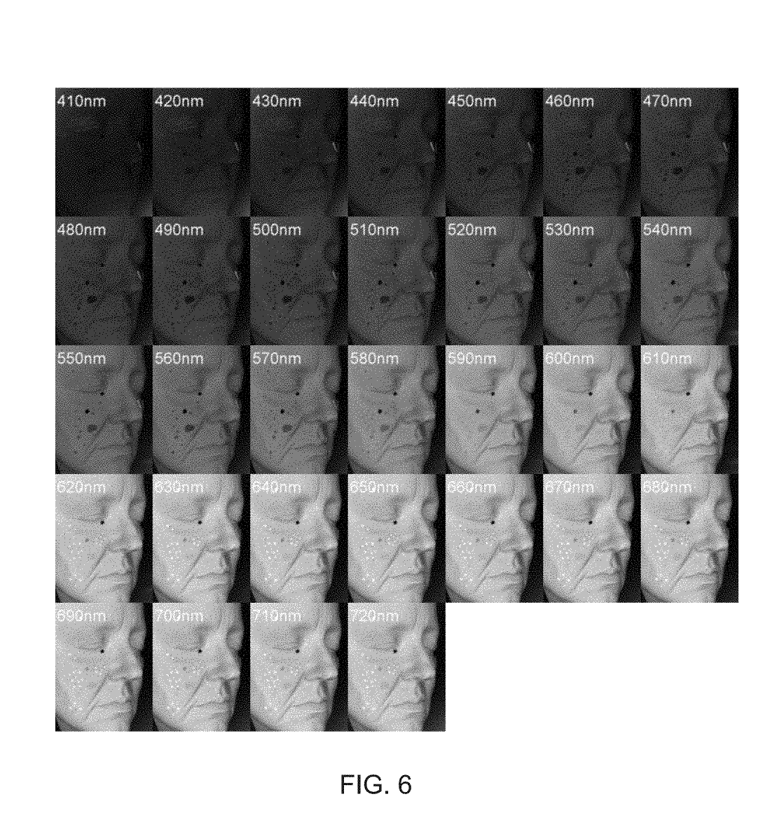

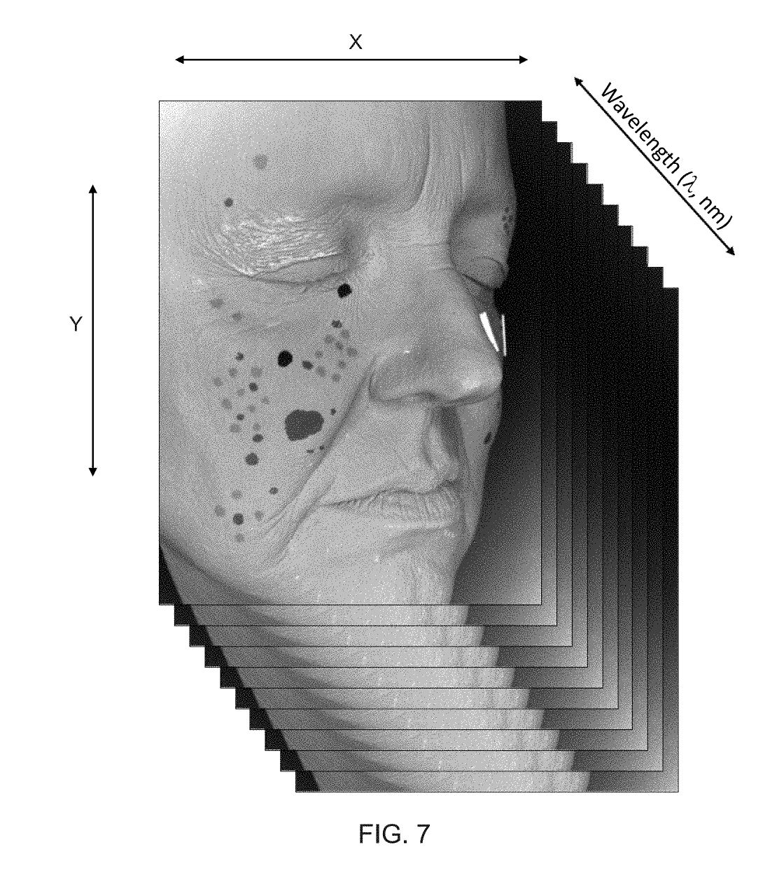

The image acquisition control component may be configured to actuate one or more components of the hyperspectral imaging system to facilitate image capture. For example, the image acquisition control component may enable a user to actuate the image capture device remotely. In another example, the image acquisition control component may be configured (e.g., via control logic) to automatically adjust the shutter speed of the camera for each wavelength at which an image is captured. In a further example, the image acquisition control component may cause the captured images (raw or processed) to be displayed on a display device such as a computer monitor. The images may be displayed individually, for example as illustrated in FIG. 6, and/or as a hyperspectral image stack, as illustrated in FIG. 7. The control logic and/or processing logic, which is described in more detail below, may enable a user to interact with the displayed images (e.g., with a graphical user interface) by selecting one or more images for further observation, modification, and/or analysis. In still other non-limiting examples, the image acquisition component may be configured to control the total acquisition time, the wavelength setting of the tunable filter, the intensity of the illumination source, and/or the position of the image capture device on the positioning system. Methods of configuring hardware and software to control the devices and components described herein are known to those skilled in the art.

The hyperspectral imaging system herein may include an image processing component for analyzing, organizing, and/or modifying an image captured by the image capture device. For example, the image processing component may include hardware, firmware, and/or software for standardizing and/or calibrating raw hyperspectral image stacks, converting calibrated spectral image stacks into one or more RGB, Lab, LCh, and/or XYZ color space images at a desired lighting condition (e.g., D65/10, D55/10, D65/2, D55/2, F2, F7, TL84). Some other non-limiting lighting conditions that may be suitable are described in ASTM E308. Converting a spectral image to a color space image (e.g., LCh) may be done using conventional methods, which are known to those skilled in the art. The image processing component software may include processing logic that causes a computer to perform the desired operation (i.e., organize, analyze, and/or modify) on a captured image. The processing logic may be stored on the same or different storage medium as the control logic. The image processing component hardware may include circuitry and/or other computing infrastructure, as desired.

The hyperspectral imaging system may optionally include diagnostic logic to analyze a processed or raw image to determine the presence and/or severity of a skin condition, vascular condition, hair condition, or periorbital dyschromia condition. In some instances, the analysis may include identifying a particular feature in the image (e.g., a face or facial feature such as nose, mouth, forehead, or eye(s)), extracting data from the image, registering an object in the image, and/or normalizing a feature in the image using a common coordinate system.

In some instances, the diagnostic logic may cause a captured image of a person to be analyzed to determine a severity score that corresponds to the severity of a skin condition identified in the captured image. The image processing logic may also cause a percentile for the severity of the condition to be determined by comparing the severity of a test subject's condition to data associated with a population of people who share at least one common characteristic with the test subject. The population data used may be specific to the analyzed person's age, gender, Fitzpatrick skin type, geographic location, ethnic origin, or any other factor. For example, if 55% of a sample group of people in the analyzed person's age group had a severity score for the skin condition that is below the analyzed person's severity score, and 45% of the sample group had a severity score above the analyzed person's severity score, then a percentile of 55 or 56 is determined.

Some non-limiting examples of suitable diagnostic logic and algorithms for use therein are described in Japanese Patent Document 95-231883; “Skin Surface Analysis System and Skin Surface Analysis Method,” PCT Document WO 98/37811, “Systems and Methods for the Multispectral Imaging and Characterization of Skin Tissue,” and U.S. Pat. No. 5,016,173, “Apparatus and Method for Monitoring Visually Accessible Surfaces of the Body.” Other non-limiting examples of image processing and analysis techniques can be found in U.S. Publication Nos. 2017/0272741, 2017/0270349, 2017/0270691, 2017/0270350, and 2017/0270348 and U.S. application Ser. No. 15/465,166.

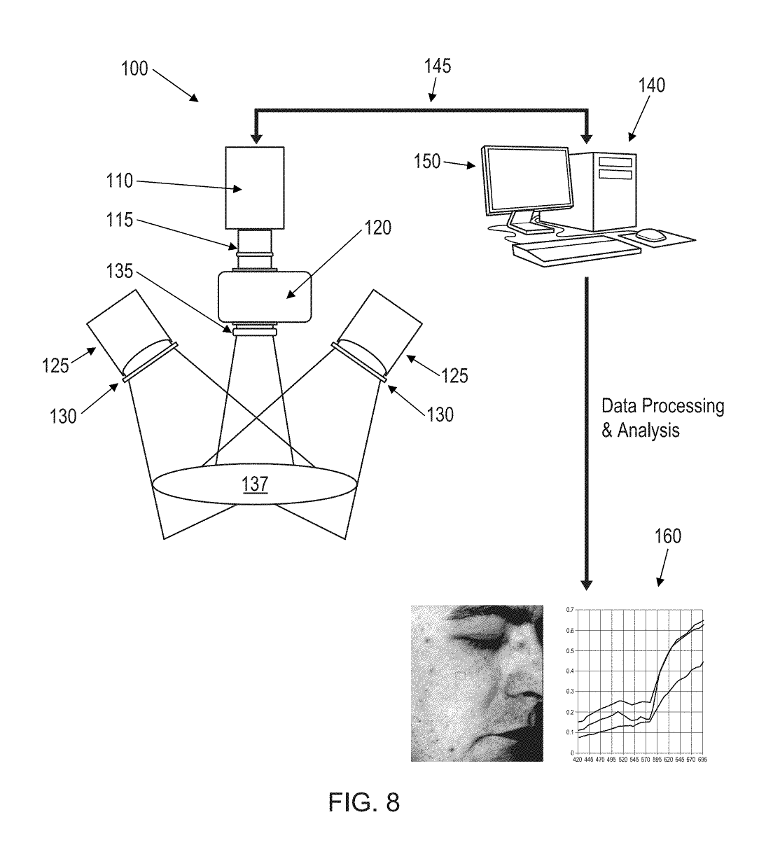

FIG. 8 illustrates an example of a hyperspectral system 100. The system 100 in this example includes an image capture device 110, a lens 115 joined to the image capture device 110, and a tunable filter 120 joined to the lens 115. An IR cutoff filter 135 is positioned between the tunable filter 120 and a target object 137 to be photographed. The system 100 includes an illumination source. In this example, the illumination source is configured as a pair of lights 125 positioned on opposite sides of the tunable filter 120 and directed toward the target object 137. As illustrated in FIG. 8, each light 125 includes a polarizer 130 to cross-polarize light emitted between the light sources 125 and the polarizer of the tunable filter, if included. In use, light from a light source 125 passes through the polarizer 130 and strikes the target object 137. At least some of the light is reflected off the target object 137 and passes through the IR cutoff filter 135 and then the tunable filter 120. The filtered light is received by the imaging sensor of the image capture device 110, which generates a digital image corresponding to the wavelength of the light transmitted through the tunable filter 120.

As illustrated in FIG. 8, the image capture device 110 is electronically coupled to a computer 140 via a network connection 145, which may be wired, wireless, or a combination thereof. In this example, the computer 140 includes a non-transitory, computer-readable storage medium that can store control logic and processing logic. The control and/or image processing logic may cause the computer 140 to receive, store, analyze, modify, and/or display spectral images received from the image capture device 110. In some instances, the control logic may communicate with and/or control one or more components of the system 100. For example, the control logic may cause the image capture device 110 to capture one or more spectral images based on a predetermined pattern of varying shutter speeds. The control logic may tune the tunable filter 120 to a desired wavelength setting and the cause the image capture device to capture a spectral image at that wavelength setting. In some instances, the processing logic and/or control logic may communicate with a user via a monitor 150. For example, the control and/or processing logic may cause the monitor 150 to display image analysis results 160 generated by the processing logic.

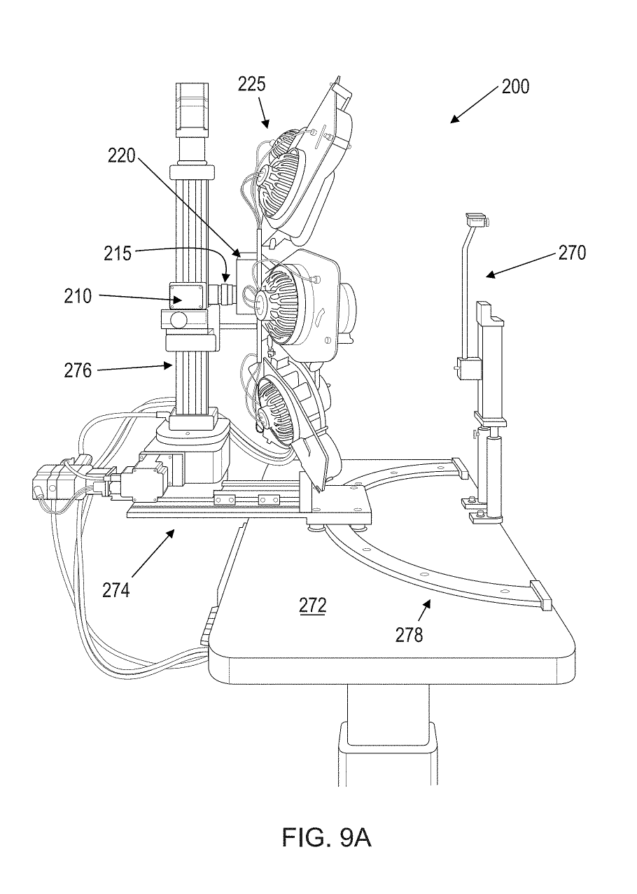

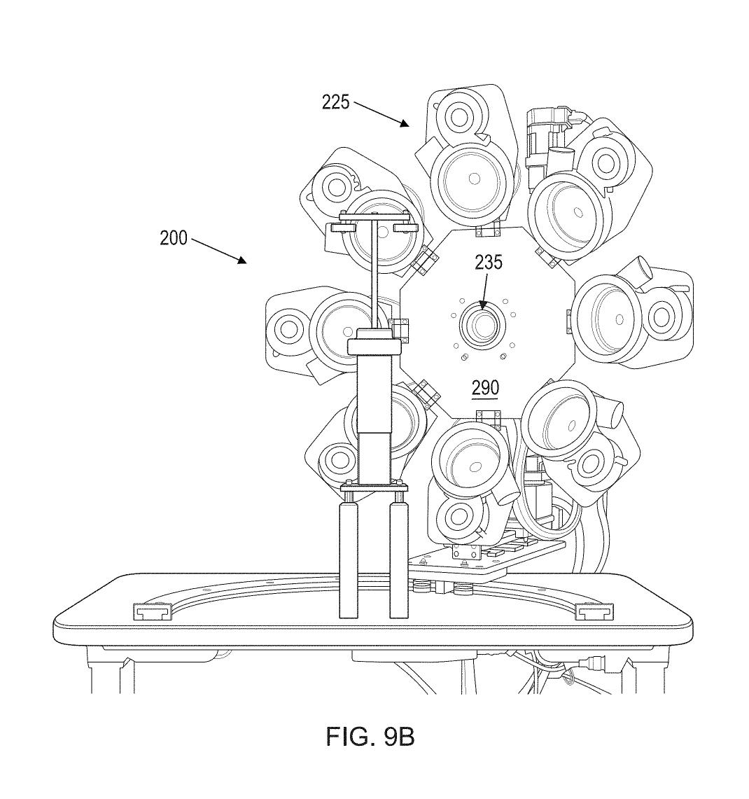

FIGS. 9A and 9B illustrate another example of a hyperspectral imaging system 200. FIG. 9A shows a side view of the exemplary system 200 and FIG. 9B shows a front view thereof. The system 200 illustrated in this example includes an image capture device 210, a lens 215 joined to the image capture device, and a tunable filter 220 joined to the lens. An IR cutoff filter 235 is positioned in front of the tunable filter 220. The system 200 also includes an illumination source comprising eight lights 225. As illustrated in FIGS. 9A and 9B, the system 200 includes a mounting component comprising a stable, level surface 272, a chin rest 270 joined to the surface 272, and a mounting element 274 joined to the surface 272. The mounting element 274 includes a vertical positioning element 276 and a circumferential positioning element 278. The image capture device 210, lens 215, tunable filter 220, IR cutoff filter 235, and lights 225 are joined to the mounting element 274 directly or indirectly via frame 290. The vertical and circumferential position elements 276 and 278 enable a user to stably reposition the system components joined thereto. The adjustable position enables a user to capture images of skin conditions or defects that are more readily visible from a particular position (e.g., skin defects that are only present on one side of the face) without the need to reposition the test subject.

### Method of Use

When capturing an image of a test subject in a clinical setting, it can be important for the test subject to remain still to help ensure the captured image is of sufficient quality (e.g., no blur), which can be challenging when capturing a large number of images (e.g., greater than or equal to 10, 20, 30, 40, or 50) for hyperspectral analysis. Thus, the fast acquisition time, spectral and spatial resolution, large image acquisition area, and high throughput of the present system make it especially suitable for use in a clinical setting.

The present system may be used to analyze a captured image, for example, using the processing logic described above. In some instances, the analysis may include evaluating a skin condition, a vascular condition (e.g., degree of oxygenation, deoxygenation, and/or other blood related condition), periorbital dyschromia (a.k.a. undereye dark circles), and/or the presence, absence, or condition of hair (e.g., shave stubble or stray hair). For example, the present system may be used to determine whether a particular skin condition is present, the severity of a skin condition, and/or changes in a skin condition over time. In another example, the present system may be used to measure and/or evaluate a cosmetic composition that has been topically applied to skin. For example, the system may be used to determine the amount of composition that is initially applied, how much composition stays on the skin over time, how much of the composition remains after cleansing, and/or the efficacy of a composition. In another example, the system may be used to determine a condition of skin and/or hair before and/or after a shaving event (e.g., presence or absence of hair stubble or stray hairs, presence or severity of skin irritation, and/or amount of shave prep composition present on the skin).

It can be important to calibrate the system prior to use to help ensure that the images captured by the system accurately depict the target object to be photographed. The system may be calibrated daily, prior to each use, or at any other suitable periodicity, as desired. Image analysis generally relies on reflectance values generated by the imaging sensor of the image capture device. It is not uncommon for these values to be instrument specific. For example, the reflectance value may be different for different digital cameras. Accordingly, it would be desirable to correlate the color response of the image capture device to a reflectance standard. Calibration may be done according to any suitable calibration method known in the art. For example, the image may be calibrated with a known standard gray scale chart (e.g., a KODAK brand Gray Scale Q-13 calibration chart, a MUNSELL brand gray scale chart, or a MUNSELL brand neutral color chart). This step includes a mathematical regression (e.g., linear regression) to correlate the captured image to the known standard (i.e., gray scale chart), which can be readily performed by those skilled in the art. Some other non-limiting examples of calibration techniques that may be suitable for use herein are disclosed in Kohler, et al. “New Approach for the Radiometric Calibration of Spectral Imaging Systems,” Optics Express Vol. 12, No. 11, p. 2463 (2004); Geladi, et al., “Hyperspectral imaging: calibration problems and solutions,” Chemometrics and Intelligent Laboratory Systems, 72, pp. 209-217 (2004); and Gorretta, et al., “Hyperspectral Imaging System Calibration Using Image Translations and Fourier Transform,” J. Near Infrared Spectrosc. 16, pp. 371-380 (2008).

In some instances, calibration may occur in two stages, which are referred to herein as the standardization stage and the uniformity correction stage. In the standardization stage, variation in exposure time is corrected for, since it is not uncommon for a camera to have variable shutter times. In the standardization stage, one or more calibration chips (e.g., gray scale or color chips available from Munsell Color, Grand Rapids Mich.) are included in the captured image (e.g., at the bottom, top or side of the image frame) to create one or more “regions of interest” in each image. An algorithm is determined (e.g., using known regression techniques) to mathematically transform equivalent regions of interest (for each calibration chip at a desired spectral band) to be as close to identical values as possible in every spectral image captured during testing (e.g., throughout an entire day of testing in a clinical setting). This is accomplished by applying the algorithm to at least some, and preferably all, of the pixels in the spectral image to create an exposure corrected image.

The uniformity correction stage can be used to correct for variations in lighting, filter properties, and/or lens properties that introduce non-uniformity into an image. A calibration algorithm is developed (e.g., using known linear or polynomial regression techniques) for at least some, and preferably all, of the pixels in a spectral image by taking a series of full frame gray scale images having known reflectance values. This algorithm is then applied to a spectral image to appropriately adjust the reflectance value of each pixel, resulting in a calibrated, uniform image.

After calibration, the system can be configured to capture spectral images of a test subject or target object at the desired wavelengths. Because the system will capture at least 10 spectral images and the total acquisition time is low (e.g., less than 5 seconds), it may be desirable to configure the system to automatically actuate the image capture device, control the shutter speed on the image capture device, tune the filter to the desired wavelengths, and/or store the captured images, for example, using a control and/or processing component, as described hereinabove. After the system is configured to capture the spectral images, the target object is positioned at a suitable distance and angle from the image capture device. Once the test subject or target object is suitably positioned, the system is activated and the spectral images are captured. The captured images may then be processed and analyzed, for example, using the processing component described hereinabove.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hyperspectral imaging system, comprising:
a) an image capture device positioned to capture an image of a target object;
b) an illumination component configured to illuminate the target object with a sufficient amount of light for the hyperspectral imaging system to generate a hyperspectral image of the target object;
c) a tunable filter; and
d) an infrared cut-off filter positioned between the target object and the tunable filter.

2. The hyperspectral imaging system of claim 1, wherein the tunable filter is a liquid crystal tunable filter that is tunable to at least 10 different wavelengths across a spectral range of about 380 nm to about 750 nm.

3. The hyperspectral imaging system of claim 1, wherein the tunable filter has a spectral resolution of about 1 nm to about 50 nm.

4. The hyperspectral imaging system of claim 1, further comprising an image acquisition control component that controls at least one function selected from image capture device actuation, image capture device exposure time, tunable filter wavelength selection, position of the image capture device relative to the target object, and generation of a hyperspectral image stack.

5. The hyperspectral imaging system of claim 1, wherein the illumination component includes a polarizing filter.

6. The hyperspectral imaging system of claim 5, wherein the polarizing filter of the illumination component is configured to cooperate with a polarizing filter contained within the tunable filter to cross polarize light entering the image capture device.

7. The hyperspectral imaging system of claim 1, wherein the system has a total acquisition time of about 5 seconds or less.

8. The hyperspectral imaging system of claim 1, further comprising an image processing component that converts the hyperspectral image into at least one of an RGB, Lab, LCh, and XYZ color space image.

9. The hyperspectral imaging system of claim 1, further comprising diagnostic logic that determines at least one of a presence of a skin condition, a severity of a skin condition, a change in a skin condition, a presence of a skin care composition, and a change in an amount of a skin care composition present on skin based on an analysis of a captured image.

10. The hyperspectral imaging system of claim 9, wherein the diagnostic logic determines a severity of a skin condition and generates a percentile score by comparing the severity of the skin condition to data associated with a population of people sharing a common characteristic with the person, the common characteristic being selected from age, ethnicity, geographic location, and combinations of these.

11. A method of generating a hyperspectral image, comprising:
a) illuminating the target portion of skin with an illumination component;
b) filtering light reflected from the target portion of skin with an infrared cut-off filter, wherein the infrared cut-off filter attenuates light intensity at wavelengths of between 700 nm and 730 nm;
c) filtering the filtered light from (b) with a liquid-crystal tunable filter that is tunable to at least 10 different spectral bands between 400 nm and 730 nm;
d) capturing the light from (c) with an image capture device;
e) generating a spectral image of the target portion of skin using the captured light from (d);
f) repeating steps (a) to (e) to generate 10 or more spectral images at different wavelengths; and
g) displaying on a display device the 10 or more spectral images as a hyperspectral image.

12. The method of claim 11, further comprising calibrating the hyperspectral imaging system to correct for at least one of variations in exposure time, lighting, filter properties, and lens properties.

13. The method of claim 12, wherein calibrating the hyperspectral imaging system includes performing a standardization step comprising creating one or more regions of interest from one or more calibration chips in a captured spectral image, creating an algorithm from a known reflectance value for each region of interest, and adjusting reflectance values of at least some of the pixels in a captured spectral image using the algorithm.

14. The method of claim 12, wherein calibrating the hyperspectral imaging system includes performing a uniformity correction step comprising creating one or more regions of interest from a gray-scale imaging chart, creating an algorithm from a known reflectance value for each region of interest, and adjusting reflectance values of at least some of the pixels in a captured spectral image using the algorithm.

15. The method of claim 11, wherein steps (a) to (f) are completed in five seconds or less.

16. A method of analyzing a hyperspectral image to determine a characteristic of skin, comprising:
a) generating a hyperspectral image of a target portion of skin of person according to the method of claim 11;
b) analyzing the hyperspectral image with diagnostic logic that causes a computer to determine at least one of a presence of a skin condition, a severity of a skin condition, a change in a skin condition, a presence of a skin care composition, and a change in an amount of a skin care composition present on skin; and
c) communicating a result of the determination in (b) to a user.

17. The method of claim 16, wherein the diagnostic logic determines a severity of a skin condition based on analysis of the hyperspectral image.

18. The method of claim 17, wherein the diagnostic logic determines a percentile for the severity of the skin condition by comparing the severity of the skin condition to data associated with a population of people who share a common characteristic with the person.

19. The method of claim 16, wherein the target portion of skin includes facial skin.
